(19) United States
(12) Patent Application Publication
(10) Pub. No.: US 2003/0219391 A1
(43) Pub. Date: Nov. 27, 2003

(54) Dispersed powders providing ultraviolet light protection, suitable for use in cosmetic compositions

(75) Inventors: Terry Van Liew, Cranford, NJ (US); Elisa Burdzy, Milford, NJ (US); Michael Smith, Branchburg, NJ (US)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/365,653

(22) Filed: Feb. 13, 2003

Related U.S. Application Data
(60) Provisional application No. 60/359,902, filed on Feb. 28, 2002.

(51) Int. Cl. A61K 7/42

(52) U.S. Cl. 424/59

(57) ABSTRACT

The present invention provides compositions which protect the skin from the harmful effects of ultra violet radiation, which compositions have an absorbent powder dispersed with nanoparticles absorbed therein.

### FIELD OF THE INVENTION

The present invention pertains to sunscreen compositions.

### BACKGROUND OF THE INVENTION

Unprotected exposure to sun can have serious consequences.

It is well-known that irradiation of light having wavelengths between 280 nm and 320 nm (UV-B radiation) causes erythema and burning of the skin. It is also well-known that wavelengths between 320 nm and 400 nm (UV-A radiation), which tans the skin, also adversely affects it. This is particularly true for sensitive or skin that is exposed to solar radiation for long periods of time. UV-A rays cause, in particular, a premature ageing of the skin, including loss in the elasticity of the skin and the appearance of lines and wrinkles. UV-A also triggers or enhances (depending on the individual) the erythematous reaction and may even be the source of phototoxic or photoallergic reactions.

Thus, it is highly desirable to screen out both UV-B and UV-A radiation. This may be achieved by using a cosmetic or dermatological product having an adequate SPF (“Sun Protection Factor”) before exposure to sunlight: the higher the SPF value, the more protection is provided to the user.

Makeup claiming varying SPF levels is sold all over the world. These are typically photo-protective/sunscreen oil-in-water (O/W) emulsions containing, in varying concentrations, one or more standard lipophilic and/or hydrophilic organic sunscreen agents capable of selectively absorbing harmful UV radiation. It has long been desirable, however, particularly in Asia, to have a foundation with a high SPF.

However, typically foundations come in the form of pressed powders, which are difficult to make cosmetically acceptable with a high SPF. The levels of ultrafine titanium dioxide required for high SPF values result in a product that is, among others, too dry. Adding organic sunscreen agents to pressed powder compositions creates a product exhibiting a cosmetically undesirable glaze. Using a combination of both titanium dioxide and an organic sunscreen agent does not result in a high enough SPF values. Thus, it has been necessary to limit the amount of titanium dioxide and/or organic liquid sunscreen agent present to avoid such problems, which, in turn, limits the SPF value of the products.

There accordingly remains a continuing need in the art for high SPF foundations, particularly powders. Similarly, there remains a need to compositions that can be used to formulate various topical or cosmetic compositions with high SPF values.

The present inventors have developed a new powdery dispersant which enables the addition of amounts of nanoparticles (inorganic sunscreen agents) into topical or cosmetic compositions, in particular, pressed powder compositions, sufficient to obtain high SPF value products that do not suffer from the negative side effects of previous compositions; chalkiness, grittiness, dryness, glaze.

### SUMMARY OF THE INVENTION

Accordingly, one object of the present invention is to provide an absorbent powder having nanoparticles absorbed therein useful in providing sunscreen protection. This material is sometimes referred to herein as “absorbent powder/nanoparticles.” Another object of the invention is to provide sunscreen topical compositions (i.e., a cosmetic or pharmaceutical composition).

Another object of the present invention is to provide foundations providing sunscreen protection.

Another object of the present invention is to provide pressed powders providing sunscreen protection.

Another object of the present invention is to provide methods of preparing the absorbent powder with the nanoparticles absorbed therein.

Another object of the invention is to provide methods for incorporating these absorbent powders containing nanoparticles into topical or cosmetic compositions, in particular pressed powder compositions.

Another object of the invention is to provide long-lasting cosmetic and topical compositions which provide sunscreen protection.

Another object of the present invention is to provide methods of reducing, attenuating, or preventing the effects associated with sun exposures, such as fine lines and wrinkles, darkening of the skin, and/or reducing the risk of skin cancers.

### DETAILED DESCRIPTION OF THE INVENTION

The present invention provides an absorbent powder having nanoparticles absorbed therein which provide a sunscreen benefit. The present invention further provides topical compositions containing an ultraviolet light protective amount of this absorbent powder. These topical compositions include any composition that can be applied to the skin, including, particularly facial skin, such as the skin around the eyes, cheeks and lips. Examples of such compositions include pressed face powders, loose face powders, powder blushes, powder eye shadows, color corrector powders, bronzing powders, talcum powders (pressed or loose and fragranced or un-fragranced), liquid talcum powders, aerosol powders, aerosol compositions (fragranced or un-fragranced) anhydrous systems (including cream to powders, stick compositions, such as make-up, blush, shimmer sticks, bronzing compositions, eye shadows, concealers, and lipsticks), emulsions of any kind (silicone emulsions, O/W, W/O and multiple emulsions, all of which may be pigmented or un-pigmented), under eye treatment compositions, nail care compositions, leave-on-hair compositions (such as conditioners, etc.), dispersions (aqueous or non-aqueous, such as silicone dispersions, oil dispersions), suspensions (aqueous or non-aqueous, such as silicone or oil suspensions), deodorants, anti-perspirants (that may be molded in tubes or poured), lip products (including lip coating products, such as top or bottom coats, lipsticks, lip gloss, lip paint, lip coating, lip powder, lip liners, lip protection/anti-chapping sticks, lip plumping, and lip pencils).

In another embodiment, the present invention provides long-wear pressed powder, foundation, or lipstick compositions having an SPF of at least 18. Long-wear is defined herein as being a composition that last up to 6 hours after application to the skin or lips, including up to 8 hours.

The powder compositions containing the absorbent powder/nanoparticles dispersion of the present invention, when applied to the skin, do not result in a chalky, dry, gritty appearance on white or non-white skin. The term “chalky” refers to a white powdery residue that is left on the skin after application of a composition to the skin. The term “gritty” refers to residues left on the skin which feel rough or gritty to the user.

The term “non-white pigmented skin” refers to the tone of human skin, ranging from light yellow to dark brown. Non-white pigmented skin is commonly associated with, for example, Asians, Indians, American Indians, Hispanics and African Americans. Such skin is also associated with individuals whose skin has darkened from exposure to the sun, tanning machines or skin coloring agents.

In one embodiment the absorbent powder is silica. Silica suitable for use in the invention includes a porous non-fumed silica, such as porous spherical non-fumed silica. The silica has a particle size in the range of 0.5 to 20 μm, including 1 to 15 μm, and including 1 to 5 μm. In another embodiment the individual silica particles are 0.5 to 1.0 μm in size and exist as agglomerates of 5 to 7 μm in diameter. In addition, the silica should be highly porous and have a pore volume of 0.1 to 10 ml/g, including 1 to 5 ml/g. Typically, the silica has a surface area of about 300 to 1000 m²/g, including 600 to 800 m²/g. Further, suitable silica for use in the invention should have an absorptive activity of 200 to 1000 g, including 300 to 800 g, including 600 to 700 g of nanoparticles to 100 g of silica.

Silica shells suitable for use in the present invention are those described in U.S. Pat. No. 5,024,826, the entire content of which is incorporated herein by reference. For example, silica for use in the powder compositions according to the invention is MSS 500/3H, a highly porous silica available from Kobo Products, Inc., South Plainfield, N.J.

In one embodiment the nanoparticles are inorganic compounds composed essentially of metal oxides. Suitable metal oxides comprise one or more of iron oxide, aluminum oxide, zirconium oxide, vanadium oxide, niobium oxide, tantalum oxide, chromium oxide, molybdenum oxide, tungsten oxide, cobalt oxide, nickel oxide, cerium cupric oxide, zinc oxide, tin oxide, antimony oxide titanium dioxide and mixtures thereof, among others. In yet another embodiment titanium dioxide and zinc oxide are used. Without being limited to theory, in most cases the metal oxide nanoparticles provide a sun protection benefit by diffracting the ultraviolet light. The elemental size of 1 nanoparticle is typically from less than 1 μm in size, including from about 100 nm to about 500 nm, including about 200 nm to about 350 nm. Preferably, the size of the nanoparticles is less than the size of the void in the absorbent powders

The nanoparticles are absorbed into the absorbent powders by adding the nanoparticles suspended or dispersed in a solvent and blending the mixture until the nanoparticles are absorbed into the absorbent powders. Absorption of the nanoparticles can be assessed by observing the physical nature of the mixture, for example, when the mixture appears to form a free flowing powder the nanoparticles are sufficiently absorbed into the absorbent powders. Additional methods are described herein. In one embodiment the nanoparticles are coated prior to absorption into the absorbent powders. Suitable coatings include, but are not limited to, stearic acid, amino acids, silicones, aluminum starch octenyl succinate isopropyl titanium triisostearate, perfluoropolyethers, fluorinated compounds, aluminum hydroxide, hydrogenated lecithin, metal soaps, oils, glycerol rosinate, esters, polyethylene, active lipid plant extracts, cellulose, clay, acrylate copolymer, plant waxes, lauroyl lysine, boron nitride and blends of the above at a level from 1 to 20%.

In other embodiments the nanoparticles are combined in amounts sufficient to provide as much absorption of the nanoparticles in the absorbent powder as possible. This may be achieved by combining from 10 to 1 parts of the nanoparticles to 1 part absorbent powder, including from 7 to 2 parts of the nanoparticles to 1 part absorbent powder, and including 5 to 6 parts of the nanoparticles to 1 part absorbent powder. The resulting mixture is a dry, non-agglomerated, sheer powder impregnated with the nanoparticles.

Suitable solvents to disperse the nanoparticles for use to provide into the absorbent particles include, for example, cosmetic oils that are liquid at room temperature and may be selected from silicone oils and fatty acid esters. Examples of suitable solvents include dimethicone, cyclomethicone, capric/caprylic triglyceride, and isononyl isonanoate. Other suitable solvents are those found on pages 1786-188 of the International Cosmetic, Toiletries, and Fragrance Association Handbook, 8^{th} Edition (2000).

In one embodiment the absorbent particles may have one or more organic sunscreen agents absorbed therein in addition to nanoparticles. This can be achieved by combining the absorbent powder with nanoparticles coated with one or more organic sunscreen agents, or by mixing the organic sunscreen with the nanoparticles prior to absorption, or by mixing into the composition separate absorbent powders having the organic sunscreen agents absorbed therein, as described in U.S. Pat. No. 5,904,918. U.S. Pat. No. 5,904,918 describes the preparation of silica and organic sunscreen aggregates useful for providing enhanced sun protection in cosmetic powder compositions.

In an alternate embodiment, when the absorbent powders having the nanoparticles absorbed therein are formulated into a cosmetic or pharmaceutical topical composition, the organic sunscreens may be added directly into the formulation according to procedures known in the art.

Sunscreens according to the present invention which are physical blockers reflect or scatter ultraviolet radiation. Typical examples of physical blockers include red petrolatum, titanium dioxide, and zinc oxide. These physical blockers have been employed in a variety of suspensions and particle sizes and are frequently included in cosmetic formulations. A review of physical blockers may be found at “Sun Protection Effect of Nonorganic Materials,” by S. Nakada & H. Konishi, Fragrance Journal, Volume 15, pages 64-70 (1987), which is incorporated by reference herein.

Sunscreens according to this invention which are chemical absorbers, like avobenzone, actually absorb harmful ultraviolet radiation. It is well known that chemical absorbers are classified, depending on the type of radiation they protect against, as either UV-A or UV-B absorbers. UV-A absorbers generally absorb radiation in the 320 to 400 nm region of the ultraviolet spectrum. UV-A absorbers include anthranilates, benzophenones, and dibenzoyl methanes. UV-B absorbers generally absorb radiation in the 280 to 320 nm region of the ultraviolet spectrum. UV-B absorbers include p-aminobenzoic acid derivatives, camphor derivatives, cinnamates, and salicylates.

Classifying the chemical absorbers generally as UV-A or UV-B absorbers is accepted within the industry. However, a more precise classification is one based upon the chemical properties of the sunscreens. There are eight major classifications of sunscreen chemical properties which are discussed at length in “Sunscreens—Development, Evaluation and Regulatory Aspects,” by N. Shaath et al., 2nd. Edition, pages 269-273, Marcel Dekker, Inc. (1997). This discussion, in its entirety, is incorporated by reference herein.

The sunscreens which may be formulated according to the present invention typically comprise chemical absorbers, but may also comprise physical blockers. Exemplary sunscreens which may be formulated into the compositions of the present invention are chemical absorbers such as p-aminobenzoic acid derivatives, anthranilates, benzophenones, camphor derivatives, cinnamic derivatives, dibenzoyl methanes, diphenylacrylate derivatives, salicylic derivatives, triazine derivatives, benzimidazole compounds, bis-benzoazolyl derivatives, methylene bis-(hydroxyphenylbenzotriazole) compounds, the sunscreen polymers and silicones, or mixtures thereof. These are variously described in U.S. Pat. Nos. 2,463,264, 4,367,390, 5,166,355 and 5,237,071 and in EP-0,863,145, EP-0,517,104, EP-0,570,838, EP-0,796,851, EP-0,775,698, EP-0,878,469, EP-0,933,376, EP-0,893,119, EP-0,669,323, GB-2,303,549, DE-1,972,184 and WO-93/04665, also expressly incorporated by reference. Also exemplary of the sunscreens which may be formulated into the compositions of this invention are physical blockers such as cerium oxides, chromium oxides, cobalt oxides, iron oxides, red petrolatum, silicone-treated titanium dioxide, titanium dioxide, zinc oxide, and/or zirconium oxide, or mixtures thereof.

A wide variety of sunscreens is described in U.S. Pat. No. 5,087,445, issued to Haffey et al. on Feb. 11, 1992; U.S. Pat. No. 5,073,372, issued to Turner et al. on Dec. 17, 1991; and Chapter VIII of Cosmetics and Science and Technology by Segarin et al., pages 189 et seq. (1957), all of which are incorporated herein by reference in their entirety.

Sunscreens which may be formulated into the compositions of the instant invention are those selected from among: aminobenzoic acid, amyldimethyl PABA, cinoxate, diethanolaminep-methoxycinnamate, digalloyl trioleate, dioxybenzone, 2-ethoxyethyl p-methoxycinnamate, ethyl 4-bis(hydroxypropyl)aminobenzoate, 2-ethylhexyl-2-cyano-3,3-diphenylacrylate, ethylhexyl p-methoxycinnamate, 2-ethylhexyl salicylate, glyceryl aminobenzoate, homomenthyl salicylate, homosalate, 3-imidazol-4-ylacrylic acid and ethyl ester, methyl anthranilate, octyldimethyl PABA, 2-phenylbenzimidazole-5-sulfonic acid and salts, red petrolatum, sulisobenzone, titanium dioxide, triethanolamine salicylate, N,N,N-trimethyl-4-(2-oxoborn-3-ylidene methyl)anillinium methyl sulfate, and mixtures thereof.

Sunscreens active in the UV-A and/or UV-B range can also include:

p-aminobenzoic acid,

oxyethylene (25 mol) p-aminobenzoate,

2-ethylhexyl p-dimethylaminobenzoate,

ethyl N-oxypropylene p-aminobenzoate,

glycerol p-aminobenzoate,

4-isopropylbenzyl salicylate,

2-ethylhexyl 4-methoxycinnamate,

methyl diisopropylcinnamate,

isoamyl 4-methoxycinnamate,

diethanolamine 4-methoxycinnamate,

3-(4′-trimethylammunium)-benzyliden-bornan-2-one methylsulfate,

2-hydroxy-4-methoxybenzophenone,

2-hydroxy-4-methoxybenzophenone-5-sulfonate,

2,4-dihydroxybenzophenone,

2,2′,4,4′-tetrahydroxybenzophenone,

2,2′-dihydroxy-4,4′dimethoxybenzophenone,

2-hydroxy-4-n-octoxybenzophenone,

2-hydroxy-4-methoxy-4′-methoxybenzophenone,

-(2-oxobom-3-ylidene)-tolyl-4-sulfonic acid and soluble salts thereof,

3-(4′-sulfo)benzyliden-bornan-2-one and soluble salts thereof,

3-(4′methylbenzylidene)-d,l-camphor,

3-benzylidene-d,l-camphor,

benzene 1,4-di(3-methylidene-10-camphosulfonic) acid and salts thereof (the product Mexoryl SX described in U.S. Pat. No. 4,585,597 issued to Lange et al. on Apr. 29, 1986),

urocanic acid,

2,4,6-tris[p-(2′-ethylhexyl-1′-oxycarbonyl)-anilino]-1,3,5-triazine,

2-[(p-(tertiobutylamido)anilino]-4,6-bis-[(p-(2′-ethylhexyl-1′-oxycarbonyl)anilino]-1,3,5-triazine,

2,4-bis {[4-(2-ethyl-hexyloxy)]-2-hydroxy]-phenyl}-6-(4-methoxy-phenyl)-1,3,5-triazine (“TINOSORB S” marketed by Ciba),

the polymer of N-(2 et 4)-[(2-oxoborn-3-yliden)methyl]benzyl]-acrylamide,

1,4-bisbenzimidazolyl-phenylen-3,3′,5,5′-tetrasulfonic acid and salts thereof,

the benzalmalonate-substituted polyorganosiloxanes,

the benzotriazole-substituted polyorganosiloxanes (Drometrizole Trisiloxane),

dispersed 2,2′-methylene-bis-[6-(2H-benzotriazol-2-yl)-4-(1,1,3,3-tetramethylbutyl)phenol] such as that marketed under the trademark MIXXIM BB/100 by Fairmount Chemical, or micronized in dispersed form thereof such as that marketed under the trademark TINOSORB M by Ciba-Geigy, and

solubilized 2,2′-methylene-bis-[6-(2H-benzotriazol-2-yl)-4-(methyl)phenol] such as that marketed under the trademark MIXXIM BB/200 by Fairmount Chemical. Typically combinations of one of more of these sunscreens are used.

The dibenzoyl methane derivatives other than avobenzone are described, for example, in FR-2,326,405, FR-2,440,933 and EP-0,114,607, hereby expressly incorporated by reference.

Other dibenzoyl methane sunscreens other than avobenzone include (whether singly or in any combination):

2-methyl dibenzoylmethane

4-methyldibenzoylmethane

4-isopropyldibenzoylmethane

4-tert.-butyldibenzoylmethane

2,4-dimethyldibenzoylmethane

2,5-dimethyldibenzoylmethane

4,4′-diisopropyldibenzoylmethane

4,4′-dimethoxydibenzoylmethane

2-methyl-5-isopropyl-4′-methoxydibenzoylmethane

2-methyl-5-tert.-butyl-4′-methoxydibenzoylmethane

2,4-dimethyl-4′-methoxydibenzoylmethane

2,6-dimethyl-4-tert.-butyl-4′-methoxydibenzoylmethane

Additional sunscreens that can be used are described in pages 1788 to 1789 of the International Cosmetic, Toiletries, and Fragrance Association Handbook, 8^{th} Edition (2000).

The absorbent powder/nanoparticles of the present invention may be added to a cosmetically acceptable carrier to provide a cosmetic powder composition having an enhanced sun protecting efficacy (sun protection factor-SPF) with desirable textural attributes. Such desirable attributes include an ultra-sheer, substantially oil-free powder. Such powders include: pressed or loose face powders, two-way cakes, pressed or loose powder eye shadow, pressed or loose color corrector powder, pressed or loose bronzing powder, pressed or loose talcum powder, aerosol powders, and the like.

In one embodiment, the absorbent powders having nanoparticles absorbed therein are formulated with one ore more colorants or pigments and may also contain various fillers. The term pigments” refers to white or colored, inorganic or organic particles which are insoluble in the liquid fatty phase and are intended to color and/or opacity the product. The term “fillers” refers to colorless or white, inorganic or synthetic, lamellar or non-lamellar particles. The term “nacres” refers to iridescent particles produced in particular by certain molluscs in their shell, or else synthesized. These fillers and nacres serve in particular to modify the texture of the composition.

The pigments can be present in the composition in a proportion of from 0.05 to 25% of the weight of the final composition, and preferably in a proportion of from 2 to 15%. These ranges include all specific values and subranges therebetween, such as 0.1, 0.2, 0.5, 1, 3, 5, 10, 12, 18 and 20% by weight. As inorganic pigments which can be used in the invention, mention may be made of titanium oxide, zirconium oxide or cerium oxide, as well as zinc oxide, iron oxide or chromium oxide and ferric blue. Among the organic pigments which can be used in the invention, mention may be made of carbon black and barium, strontium, calcium (DC Red No. 7) and aluminum lakes.

The nacres can be present in the composition in a proportion of from 0 to 20% of the total weight of the composition, preferably in a proportion ranging from 1 to 15%. These ranges include all specific values and subranges therebetween, such as 0.1, 0.2, 0.5,2, 5, 10 and 15% by weight. Examples of nacres which may be used in the invention include mica coated with titanium oxide, with iron oxide, with natural pigment or with bismuth oxychloride, such as colored titanium mica.

These products can be molded, poured, provided in tubes or any other means for packaging and delivering the product to the consumer. In another embodiment, the powdery dispersant of the absorbent powder and nanoparticles can be added onto or impregnated into a non-woven, woven or sponge-like substrate for delivery to the user. Such articles may consist of one or more layers, which layers may be apertured or non-apertured and the powdery dispersant can be provided admixed in a topical or cosmetic composition or may be added separately from other active or cosmetically effective agents to provide a sunscreen benefit to the user.

As desired the compositions containing the dispersed powder of the present invention can be provided with fragrances.

The absorbent powder having nanoparticles absorbed therein is present in a topical or cosmetic composition in an amount of about 3 to 40 weight percent, including about 3 to 35 weight percent, and including about 5 to 30 weight percent of the powder composition. This absorbent powder having nanoparticles absorbed therein allows for the preparation of a substantially oil-free powder with enhanced SPF efficacy. The enhanced SPF is achieved by delivering effective amounts of sunscreen in the powder via the absorbent powder having nanoparticles absorbed therein wherein high amounts of sunscreen nanoparticles are entrapped inside the absorbent powder. The absorbent powder having nanoparticles absorbed therein is effective in protecting human skin from the harmful effects of ultraviolet radiation, such as sunburn and sun-induced premature aging of the skin. Measurement of the levels of sunscreen protection or SPF is performed according to the test set forth in the Federal Register Vol. 46, No. 17, of Tuesday Jan. 27, 1981. The powdery dispersant of the absorbent powder and nanoparticles preferably yield SPF values of at least 18, including from 18 to 45 and from 22 to 30, inclusive of SPF values of 19, 20, 21, 23, 24, 25, 26, 27, 28, 29, 31, 32, 33, 34, 35, 36, 37, 38, 39, 40, 41, 42, 43, and 44.

Other conventional additives typically employed in cosmetic or topical compositions may be employed in conjunction with the present invention. Such additives include, but are not limited to one or more preservatives such as methyl paraben, butyl paraben, propyl paraben, phenoxyethanol, sulfuric acid, benzoic acid, imidazolidinyl urea and other conventional preservatives, antioxidants, emollients, skin conditioning agents (such as alpha hydroxy acids (AHA), glycolic acid, lactic acid), skin bleaching agents (such as kojic acid), artificial tanning agents, vitamins (such as vitamins A, C and E), vitamin derivatives, plasticizers, surfactants, water proofing additives, botanical extracts and fillers including polyethylene, magnesium carbonate, methylcellulose, mica, among others.

To manufacture the powdery dispersant of the absorbent powder and nanoparticles, the components of the powder compositions according to the invention are dry blended together using conventional powder blending apparatus and procedures. The nanoparticles suspended or dispersed in the absorbent powder are combined with the absorbent powder in suitable amounts and blended for a sufficient time to incorporate the nanoparticles in the absorbent powder, such a time may vary but can be determined by visual inspection of the progress of the dispersion. When the dispersion of the absorbent powder and the nanoparticles in solvent appears substantially powdery then the dispersion is complete. After obtaining the dispersion, the dispersion can be formulated into a topical or cosmetic composition according to common methods known in the art, for example, dispersions, blending and the like.

Protection from fine lines and wrinkles associated with aging and/or exposure to the sun can be effectuated by applying one or more of the compositions described herein thereby providing a sunscreen benefit, that is compared to an individual not so protected from the harmful effects of ultraviolet light. Therefore, the powdery dispersant of the present invention can be used to reduce the occurrence of fine lines and wrinkles by applying the powdery dispersant, for example, in the form of one or more of the topical or cosmetic compositions to the skin in an amount effective to provide a sunscreen benefit thereby reducing the appearance and/or generation of fine lines and/or wrinkles. Likewise, other negative effects of exposure to ultraviolet light can be reduced, prevented or attenuated such as darkening of the skin and sun-related or induced cancers, e.g., skin cancer, melanoma and the like.

In a method of reducing, preventing or attenuating UV-light-induced skin damage the powdery dispersant, with one or more of the cosmetic or topical compositions described herein, is administered in an effective amount to the skin of the user who requires or wishes to obtain benefit of the reduced, prevented or attenuated effects of UV-light. These compositions, depending on their form, may be spread, brushed, sprayed or any other suitable delivery means onto the skin or hair. The compositions of the invention may be applied in an amount of from about 0.5 to about 100 milligrams per cm² of skin; including from about 1 to about 50 milligrams per cm of skin, and including from 1 to 25 milligrams per cm² of skin. The compositions are applied prior to or during exposure to UV light. In one embodiment, the sunscreen compositions are applied at least once daily.

In another embodiment, the nanoparticles containing compositions can be applied to the face and/or skin followed by the application of other topical and/or cosmetic compositions over the nanoparticles containing compositions.

The following Examples provide an illustration of embodiments of the invention and should not be construed to limit the scope of the invention, which is set forth in the appended claims. In the following Examples, all methods described are conventional unless otherwise specified.

### EXAMPLES

### Example 1

A silica/titanium dioxide dispersion was prepared by mixing the following in a Kitchen Aid blender, which was then added to a CBM module and further mixed for 10 minutes at 3000 RPM:

75% by weight of a dispersion (composed of 45%, Isononyl Isonanoate 37 to 43% Titanium Dioxide, 3.3 to 5.5% Stearic acid, 5.5% dimethicone, and 3.8 to 6.1% aluminum hydroxide); and 25% by weight of porous silica MSS 500-3H (obtained from Kobo Products, Inc., South Plainfield, N.J.).

### Example 2

15 The preparation of Example 1 was incorporated into a composition as follows:

| Phase | Material | % |
|---|---|---|
| A | TiO₂ | 10.00 |
| | ultrafine TiO₂ and ZnO₂ | 10.00 |
| | talc | 15.99 |
| | preservatives | 1.05 |
| B | pigments | 11.81 |
| | Dow Corning 9506 | 3.25 |
| | silica | 2.50 |
| | cerium dioxide | 1.00 |
| | polyamide | 4.0 |
| C | silica titanium dioxide dispersion from Example 1 | 30.00 |
| D | Capric/Caprylic triglyceride | 0.75 |
| | organic sunscreen | 9.00 |
| | glyceryl octanoate/stearate/adipate | 1.00 |
| | Total | 100.00 |

Phase A was dispersed in a pin mill and subsequently phase B was added and blended for 10 minutes at 5000 RPM in a CBM module. Phase C was added and blended for 10 minutes at 3000 RPM in a CBM module. Phase D was added and blended for 3000 RPM for 10 minutes.

The composition will be expected to provide an SPF value of at least 18 and when applied to the skin is not chalky or gritty.

### Example 4

A silica/titanium dioxide dispersion was prepared by mixing the following in a Kitchen Aid blender, which was then added to a CBM module and further mixed for 10 minutes at 3000 RPM: 83.34% by weight of a dispersion (composed of 45%, Isononyl Isonanoate 37 to 43% Titanium Dioxide, 3.3 to 5.5% Stearic acid, 5.5% dimethicone, and 3.8 to 6.1% aluminum hydroxide); and 16.66% hollow silica shells.

### Example 5

The silica/titanium dioxide dispersion from Example 4 was formulated into a composition as follows:

| Phase | Material | % |
|---|---|---|
| A | TiO₂ | 10.00 |
| | ultrafine TiO₂ and ZnO₂ | 10.00 |
| | talc | 18.90 |
| | preservatives | 0.35 |
| B | pigments | 10.75 |
| | magnesium stearate | 1.00 |
| | silica | 5.00 |
| | cerium dioxide | 1.00 |
| | polyamide | 4.0 |
| C | silica titanium dioxide dispersion from Example 4 | 30.00 |
| D | Capric/Caprylic triglyceride | 1.00 |
| | organic sunscreen | 7.00 |
| | glyceryl octanoate/stearate/adipate | 1.00 |
| | Total | 100.00 |

Phase A was dispersed in a pin mill and subsequently phase B was added and blended for 10 minutes at 5000 RPM in a CBM module. Phase C was added and blended for 10 minutes at 3000 RPM in a CBM module. Phase D was added and blended for 3000 RPM for 10 minutes. 1.00% calcium silicate was added as needed.

The above-formulation was tested on 5 subjects for sun protection (SPF) according to the methods described in Federal Register Vol. 46, No. 17, of Tuesday Jan. 27, 1981. The SPF results averaged: 27.16. The resulting powder was a smooth, creamy powder that gave a natural appearance to the skin. It was not chalky or gritty.

### Example 6

Dispersed powders of silica and titanium dioxide (435-047) were prepared as above with the following components:

| Material | 1 | 2 | 3 | 4 |
|---|---|---|---|---|
| silica shells | 25% | 14% | | |
| HB50S4 ^{A} | 75% | 86% | 75% | 75% |
| Acrylates copolymer | | | 25% | |
| allyl methacrylate copolymer | | | | 25% |

^{A} HB50S4 contains 54.10% TiO2 43% Butyl octyl salycilate, 6.05% aluminum hydroxide, and 3.85% stearic acid

The composition will be expected to provide an SPF value of at least 18 and when applied to the skin is not chalky or gritty.

### Example 7

The dispersed powder of Example 5 was formulated into pressed powders having the following components:

| Phase | Material | % |
|---|---|---|
| A | talc | 12.64 |
| | ultrafine titanium dioxide | 5.00 |
| | mica, silica, dimethicone | 4.00 |
| | polyethylene, PTFE, synthetic wax | 1.50 |
| | aluminum starch octylsuccinate | 5.00 |
| | preservatives | 0.65 |
| | lauroyl lysine | 6.00 |
| | HDI, Trimethylol hexyl lactone crosspolymer | 3.00 |
| | silica | 2.00 |
| | zinc laurate | 1.00 |
| | pigments | 21.51 |
| | silica and titanium dioxide dispersed | 30 |
| | powder of Example 6 | |
| B | pentaerythrityl tetraoctanoate | 0.07 |
| | dimethicone, trimethylsiloxysilicate | 2.18 |
| | PCP/Hexadecene Copolymer | 1.70 |
| | Neopentyl Glycol Dioctanoate, Neotpentyl | 3.75 |
| | Glycol Diisostearate | |
| A | talc | 12.64 |
| | ultrafine titanium dioxide | 5.00 |
| | mica, silica, dimethicone | 4.00 |
| | polyethylene, PTFE, synthetic wax | 1.50 |
| | ITT treated aluminum starch octylsuccinate | 5.00 |
| | preservatives | 0.65 |
| | lauroyl lysine | 6.00 |
| | HDI, Trimethylol hexyl lactone crosspolymer | 3.00 |
| | silica | 2.00 |
| | zinc laurate | 1.00 |
| | pigments | 21.51 |
| | silica and titanium dioxide dispersed | 30 |
| | powder of Example 6 | |
| B | organic sunscreen | 7.50 |
| | Neopentyl Glycol Dioctanoate, Neotpentyl | 0.20 |
| | Glycol Diisostearate | |

These compositions will be expected to provide an SPF value of at least 18 and when applied to the skin is not chalky or gritty.

### Example 8

A silica/titanium dioxide dispersion was prepared by mixing the following in a Kitchen Aid blender, which was then added to a CBM module and further mixed for 10 minutes at 3000 RPM:

75% by weight of a dispersion (composed of 75% titanium dioxide, 10% dimethicone, 7% stearic acid and 8% aluminum hydroxide); and 25% by weight of porous silica MSS 500-3H (obtained from Kobo Products, Inc., South Plainfield, N.J.). This silica/titanium dioxide dispersion can be incorporated into the formulations of Examples 2-7, which will be expected to provide an SPF value of at least 18 and when applied to the skin is not chalky or gritty.

Obviously, numerous modifications and variations on the present invention are possible in light of the above teachings. It is therefore to be understood that within the scope of the appended claims, the invention may be practiced otherwise than as specifically described herein.

What is claimed is:

1. A topical composition comprising an amount sufficient to provide ultraviolet light protection of an absorbent powder having nanoparticles absorbed therein.

2. The topical composition of claim 1, further comprising an organic sunscreen agent.

3. The topical composition of claim 1, wherein the nanoparticles are coated.

4. The topical composition of claim 3, wherein the nanoparticles are coated with an organic sunscreen agent.

5. The topical composition of claim 3, wherein the nanoparticles are coated with one ore more compounds selected from the group consisting of stearic acid, amino acids, silicones, perfluoropolyethers, and aluminum starch octenyl succinate.

6. The topical composition of claim 1, wherein the absorbent powder comprises, absorbed therein, nanoparticles and an organic sunscreen agent.

7. The topical composition of claim 1, wherein the nanoparticles comprise one or more metal oxides.

8. The topical composition of claim 7, wherein the metal oxide is selected from the group consisting of iron oxide, aluminum oxide, zirconium oxide, vanadium oxide, niobium oxide, tantalum oxide, chromium oxide, molybdenum oxide, tungsten oxide, cobalt oxide, nickel oxide, cupric oxide, zinc oxide, tin oxide, cerium oxide, antimony oxide and titanium dioxide.

9. The topical composition of claim 8, wherein the metal oxide is titanium oxide.

10. The topical composition of claim 8, wherein the metal oxide is zinc oxide.

11. The topical composition of claim 1, wherein the absorbent particles comprise silica.

12. The topical composition of claim 11, wherein the silica is a porous non-fumed silica.

13. The topical composition of claim 11, wherein the silica has a particle size of from 0.4 to 20 μm.

14. The topical composition of claim 13, wherein the silica has a particle size of from 1 to 15 μm.

15. The topical composition of claim 13, wherein the silica has a particle size of from 1 to 5 μm.

16. The topical composition of claim 1, which upon application to human skin does not result in a chalky appearance.

17. The topical composition of claim 1, which upon application to human skin does not result in a dry appearance.

18. The topical composition of claim 1, which upon application to human skin results in a non-gritty texture.

19. The topical composition of claim 1, which provides a sun protecting factor (SPF) of at least 18

20. The topical composition of claim 19, which provides a sun protecting factor (SPF) of from 18 to 40.

21. The topical composition of claim 19, which provides a sun protecting factor (SPF) of from 22 to 30.

22. A powder for application to human skin according to claim 1.

23. The powder of claim 22, which is selected from the group consisting of a pressed face powder, a loose face powder, a powder blush, a powder eye shadow, a color corrector powder, a bronzing powder, a talcum powder, and an aerosol powder.

24. A powder for application to human facial skin according to claim 22.

25. The powder of claim 24, which is selected from the group consisting of a pressed face powder, a loose face powder, a powder blush, a powder eye shadow, a color corrector powder, a bronzing powder, a talcum powder, and an aerosol powder.

26. The topical composition of claim 1, which comprises one or more members selected from the group consisting of an anhydrous composition, an emulsion, a dispersion, and a suspension.

27. An personal care article comprising a substrate and the topical composition of claim 1.

28. The personal care article of claim 27, wherein the substrate is selected from the group consisting of a non-woven substrate, a woven substrate, a sponge substrate and mixtures thereof.

29. The topical composition of claim 1, wherein said absorbent powder is an inorganic absorbent powder.

30. The topical composition of claim 1, wherein said nanoparticles are inorganic nanoparticles.

31. A method of manufacturing a topical composition comprising absorbing nanoparticles into an absorbent powder in an amount sufficient to manufacture a topical composition which provides ultraviolet light protection.

32. The method of claim 31, further comprising absorbing an organic sunscreen agent.

33. The method of claim 31, wherein the nanoparticles are coated.

34. The method of claim 33, wherein the nanoparticles are coated with an organic sunscreen agent.

35. The method of claim 33, wherein the nanoparticles are coated with one ore more compounds selected from the group consisting of stearic acid, amino acids, silicones, perfluoropolyethers, and aluminum starch octenyl succinate.

36. The method of claim 31, wherein the nanoparticles comprise one or more metal oxides.

37. The method of claim 36, wherein the metal oxide is selected from the group consisting of iron oxide, aluminum oxide, zirconium oxide, vanadium oxide, niobium oxide, tantalum oxide, chromium oxide, molybdenum oxide, tungsten oxide, cobalt oxide, nickel oxide, cupric oxide, zinc oxide, tin oxide, cerium oxide, antimony oxide and titanium dioxide.

38. The method of claim 37, wherein the metal oxide is titanium oxide.

39. The method of claim 37, wherein the metal oxide is zinc oxide.

40. The method of claim 31, wherein the absorbent particles comprise silica.

41. The method of claim 40, wherein the silica is a porous non-fumed silica.

42. The method of claim 40, wherein the silica has a particle size of from 0.4 to 20 μm.

43. The method of claim 40, wherein the silica has a particle size of from 1 to 15 μm.

44. The method of claim 40, wherein the silica has a particle size of from 1 to 5 μm.

45. The method of claim 40, wherein said absorbent powder is an inorganic absorbent powder.

46. The method of claim 40, wherein said nanoparticles are inorganic nanoparticles.

47. A method of reducing the appearance of fine lines and wrinkles on the skin comprising applying to the skin a topical composition comprising an absorbent powder having nanoparticles absorbed therein in an amount sufficient to provide ultraviolet light protection.

48. The method of claim 47, wherein the topical composition further comprises an organic sunscreen agent.

49. The method of claim 47, wherein the nanoparticles are coated.

50. The method of claim 49, wherein the nanoparticles are coated with an organic sunscreen agent.

51. The method of claim 49, wherein the nanoparticles are coated with one ore more compounds selected from the group consisting of stearic acid, amino acids, silicones, perfluoropolyethers, and aluminum starch octenyl succinate.

52. The method of claim 47, wherein the absorbent powder comprises nanoparticles and an organic sunscreen agent absorbed therein.

53. The method of claim 47, wherein the nanoparticles comprise one or more metal oxides.

54. The method of claim 53, wherein the metal oxide is selected from the group consisting of iron oxide, aluminum oxide, zirconium oxide, vanadium oxide, niobium oxide, tantalum oxide, chromium oxide, molybdenum oxide, tungsten oxide, cobalt oxide, nickel oxide, cupric oxide, zinc oxide, tin oxide, cerium oxide, antimony oxide and titanium dioxide.

55. The method of claim 54, wherein the metal oxide is titanium oxide.

56. The method of claim 54, wherein the metal oxide is zinc oxide.

57. The method of claim 47, wherein the absorbent particles comprise silica.

58. The method of claim 57, wherein the silica is a porous non-fumed silica.

59. The method of claim 57, wherein the silica has a particle size of from 0.4 to 20 μm.

60. The method of claim 58, wherein the silica has a particle size of from 1 to 15 μm.

61. The method of claim 57, wherein the silica has a particle size of from 1 to 5 μm.

62. The method of claim 47, which upon application to human skin does not result in a chalky appearance.

63. The method of claim 47, which upon application to human skin does not result in a dry appearance.

64. The method of claim 47, which upon application to human skin results in a non-gritty appearance.

65. The method of claim 47, which provides a sun protecting factor (SPF) of at least 18

66. The method of claim 65, which provides a sun protecting factor (SPF) of from 18 40.

67. The method of claim 65, which provides a sun protecting factor (SPF) of from 22 to 30.

68. The method of claim 47, wherein said absorbent powder is an inorganic absorbent powder.

69. The method of claim 47, wherein said nanoparticles are inorganic nanoparticles.

70. An absorbent powder having nanoparticles absorbed therein in an amount sufficient to provide ultraviolet light protection.

71. The absorbent powder of claim 70, further comprising an organic sunscreen agent.

72. The absorbent powder of claim 70, wherein the nanoparticles are coated.

73. The absorbent powder of claim 70, wherein the nanoparticles are coated with an organic sunscreen agent.

74. The absorbent powder of claim 70, wherein the nanoparticles are coated with one ore more compounds selected from the group consisting of stearic acid, amino acids, silicones, perfluoropolyethers, and aluminum starch octenyl succinate.

75. The absorbent powder of claim 70, wherein the absorbent powder comprises nanoparticles and an organic sunscreen agent absorbed therein.

76. The absorbent powder of claim 70, wherein the nanoparticles comprise one or more metal oxides.

77. The absorbent powder of claim 76, wherein the metal oxide is selected from the group consisting of Iron oxide, aluminum oxide, zirconium oxide, vanadium oxide, niobium oxide, tantalum oxide, chromium oxide, molybdenum oxide, tungsten oxide, cobalt oxide, nickel oxide, cupric oxide, zinc oxide, tin oxide, cerium oxide, antimony oxide and titanium dioxide.

78. The absorbent powder of claim 77, wherein the metal oxide is titanium oxide.

79. The absorbent powder of claim 77, wherein the metal oxide is zinc oxide.

80. The absorbent powder of claim 70, wherein the absorbent particles comprise silica.

81. The absorbent powder of claim 80, wherein the silica is a porous non-fumed silica.

82. The absorbent powder of claim 80, wherein the silica has a particle size of from 0.4 to 20 μm.

83. The absorbent powder of claim 80, wherein the silica has a particle size of from 1 to 15 μm.

84. The absorbent powder of claim 80, wherein the silica has a particle size of from 1 to 5 μm.

85. The absorbent powder of claim 70, which upon application to human skin does not result in a chalky appearance.

86. The absorbent powder of claim 70, which upon application to human skin does not result in a dry appearance.

87. The absorbent powder of claim 70, which upon application to human skin results in a non-gritty texture.

88. The absorbent powder of claim 70, which upon application to human skin provides a sun protecting factor (SPF) of at least 18.

89. The absorbent powder of claim 70, which upon application to human skin provides a sun protecting factor (SPF) of from 18 to 40.

90. The absorbent powder of claim 70, which upon application to human skin provides a sun protecting factor (SPF) of from 22 to 30.

91. A powder for application to human skin according to claim 70.

92. The powder of claim 91, which is selected from the group consisting of a pressed face powder, a loose face powder, a powder blush, a powder eye shadow, a color corrector powder, a bronzing powder, a talcum powder, and an aerosol powder.

93. A powder for application to human facial skin according to claim 70.

94. The powder of claim 93, which is selected from the group consisting of a pressed face powder, a loose face powder, a powder blush, a powder eye shadow, a color corrector powder, a bronzing powder, a talcum powder, and an aerosol powder.

95. The absorbent powder of claim 70, which comprises one or more members selected from the group consisting of an anhydrous composition, an emulsion, a dispersion, and a suspension.

96. A personal care article comprising a substrate and the absorbent powder of claim 70.

97. The personal care article of claim 96, wherein the substrate is selected from the group consisting of a non-woven substrate, a woven substrate, a sponge substrate and mixtures thereof.

98. The absorbent powder of claim 70, wherein said absorbent powder is an inorganic absorbent powder.

99. The absorbent powder of claim 70, wherein said nanoparticles are inorganic nanoparticles.

100. A non-chalky pressed powder comprising nanoparticles in an amount sufficient to provide an SPF of at least 18.

101. The non-chalky pressed powder of claim 100, further comprising an organic sunscreen agent.

102. The non-chalky pressed powder of claim 100, wherein the nanoparticles are coated.

103. The non-chalky pressed powder of claim 100, wherein the nanoparticles are coated with an organic sunscreen agent.

104. The non-chalky pressed powder of claim 100, wherein the nanoparticles are coated with one ore more compounds selected from the group consisting of stearic acid, amino acids, silicones, perfluoropolyethers, and aluminum starch octenyl succinate.

105. The non-chalky pressed powder of claim 100, wherein the nanoparticles comprise one or more metal oxides.

106. The non-chalky pressed powder of claim 105, wherein the metal oxide is selected from the group consisting of iron oxide, aluminum oxide, zirconium oxide, vanadium oxide, niobium oxide, tantalum oxide, chromium oxide, molybdenum oxide, tungsten oxide, cobalt oxide, nickel oxide, cupric oxide, zinc oxide, tin oxide, cerium oxide, antimony oxide and titanium dioxide.

107. The non-chalky pressed powder of claim 106, wherein the metal oxide is titanium oxide.

108. The non-chalky pressed powder of claim 106, wherein the metal oxide is zinc oxide.

109. The non-chalky pressed powder of claim 100, which comprises silica.

110. The non-chalky pressed powder of claim 109, wherein the silica is a porous non-fumed silica.

111. The non-chalky pressed powder of claim 110, wherein the silica has a particle size of from 0.4 to 20 μm.

112. The non-chalky pressed powder of claim 110, wherein the silica has a particle size of from 1 to 15 μm.

113. The non-chalky pressed powder of claim 110, wherein the silica has a particle size of from 1 to 5 μm.

114. The non-chalky pressed powder of claim 100, which upon application to human skin does not result in a chalky appearance.

115. The non-chalky pressed powder of claim 100, which upon application to human skin does not result in a dry appearance.

116. The non-chalky pressed powder of claim 100, which upon application to human skin results in a non-gritty texture.

117. The non-chalky pressed powder of claim 100, which upon application to human skin provides a sun protecting factor (SPF) of at least 18.

118. The non-chalky pressed powder of claim 100, which upon application to human skin provides a sun protecting factor (SPF) of from 18 to 40.

119. The absorbent powder of claim 100, which upon application to human skin provides a sun protecting factor (SPF) of from 22 to 30.

120. The non-chalky pressed powder of claim 100, which is selected from the group consisting of a pressed face powder, a loose face powder, a powder blush, a powder eye shadow, a color corrector powder, a bronzing powder, a talcum powder, and an aerosol powder.

121. The non-chalky pressed powder of claim 100 which is selected from the group consisting of a pressed face powder, a loose face powder, a powder blush, a powder eye shadow, a color corrector powder, a bronzing powder, a talcum powder, and an aerosol powder.

122. The non-chalky pressed powder of claim 100, which comprises one or more members selected from the group consisting of an anhydrous composition, an emulsion, a dispersion, and a suspension.

123. A personal care article comprising a substrate and the absorbent powder of claim 100.

124. The personal care article of claim 123, wherein the substrate is selected from the group consisting of a non-woven substrate, a woven substrate, a sponge substrate and mixtures thereof.

125. The non-chalky pressed powder of claim 100, wherein said nanoparticles are inorganic nanoparticles.

126. A method of making a topical composition, comprising
a. mixing an abosorbent powder with nanoparticles for a time sufficient to absorb the nanoparticles into the absorbent powder; and
b. admixing the absorbent powder with nanoparticles absorbed therein with one or more topically acceptable ingredients.

127. A process for increasing the SPF value of a composition comprising mixing an absorbent powder having nanoparticles absorbed therein into said composition.
